(19) United States
(12) Patent Application Publication
(10) Pub. No.: US 2011/0217270 A1
(43) Pub. Date: Sep. 8, 2011

(54) METHODS FOR TREATING TUMORS AND CANCEROUS TISSUES

(76) Inventors: William Aloysius Cavanagh, III, Seattle, WA (US); Benjamin Alan Tjoa, Seattle, WA (US); Charles W. Jacob, III, Bellevue, WA (US)

(21) Appl. No.: 13/108,835

(22) Filed: May 16, 2011

Related U.S. Application Data
(63) Continuation of application No. 11/087,156, filed on Mar. 22, 2005.
(60) Provisional application No. 60/557,111, filed on Mar. 25, 2004.

(51) Int. Cl. A61K 35/12; A61P 35/00

(52) U.S. Cl. 424/93.7

(57) ABSTRACT

The invention disclosed herein relates generally to immunotherapy and, more specifically, to therapeutic methods for treating tumors and cancerous tissues by first inducing necrosis or apoptosis (e.g., cryotherapy, chemotherapy, radiation therapy, ultrasound therapy, or a combination thereof applied against at least a portion of the tumor or cancerous tissue), and then delivering one or more se doses of antigen presenting cells (e.g., autologous dendritic cells) intratumorally or proximate to the tumor or cancerous tissue, but only after a selected period of time sufficient for the bioavailability of liberated cancer-specific antigens (monitored over the selected period of time) resulting from the necrosis or apoptosis to be at or near a maximum value. The present invention provides an alternative strategy to the ex vivo loading of target antigen to antigen presenting cells such as, for example, enriched autologous dendritic cells for purposes of enhancing an immune response.

This application is a continuation of U.S. patent application Ser. No. 11/087,156, filed Mar. 22, 2005, which application claims priority under 35 U.S.C. 119(e) to provisional application Ser. No. 60/557,111, filed on Mar. 25, 2004, the entire contents of which are incorporated herein by reference in their entireties.

### TECHNICAL FIELD

The present invention relates generally to immunotherapy and, more specifically, to therapeutic methods for treating tumors and cancerous tissues by distressing tumor cells in order to liberate tumor antigen(s), and delivering antigen presenting cells, capable of exploiting the liberated antigen(s), to the tumor or cancerous tissue.

### BACKGROUND OF THE INVENTION

Cancer Immunotherapy

Interest in cancer vaccination arose based on William Coley's early observation of cancer regression after streptococcus pyogenes infection (Coley, W. B., Clin. Orthop. 1991(262):3-3-11 (1893)). Coley noted dramatic regression of some sarcoma lesions following experimental streptococcal infection of the skin (erysipelas). While discredited during his time, Coley's observations most certainly mark the beginning of the cancer immunotherapy era. Many decades later, cancer immunotherapy began to focus on elucidating the immunologic mechanism responsible for the elimination of neoplastic cells, as well as the antigenic make-up of possible cancer vaccines.

Whether directed against a pathogen or a tumor cell, antigen is the main component of a vaccine. It is the specific target against which the immune response is generated. Ideally, antigens for cancer vaccination are those proteins with high expression in cancer cells but no expression in normal cells (cancer- or tumor-specific antigens). The search for such antigens has fueled much research in the areas of immunology and molecular biology, and has lead to the concept of the tumor-associated antigen (TAA) (Robbins and Kawakami, Current Opin Immunol 8(5):628-636 (1996); Urban and Schreiber, Ann Rev Immunol 10:617-644 (1992)).

As it turns out, antigens that are expressed uniquely on tumor cells are relatively rare. Telomerase reverse transcriptase (TERT) is an example of a TAA activated in most human tumors while absent in most normal tissues (Vonderheide et al., Immunity 10(6):673-679 (1999)). In a few cases, there exist antigens unique to tumor cells due to the mutation of normal proteins, for example: mutations in p53 (Theobald et al., Proc Natl Acad Sci USA 92(26):11993-11997 (1995)) and CDK4 proteins (Wolfel et al., Science 268(5228):1281-1294 (1995)). Most TAAs, however, are proteins typically expressed on benign cells that undergo enhanced or altered expression in tumor cells. For example, carcinoembryonic antigen (CEA) is overexpressed by breast, colon, lung and pancreas carcinomas, while MUC-1 is overexpressed by breast, lung, prostate, stomach, colon, ovary and pancreas carcinomas (Wolfel et al., Science 269(5228):1281-1284 (1995)). Some TAAs are differentiation or tissue specific, e.g., tyrosinase (Brichard et al., J Exp Med 178(2):489-495 (1993)). MART-1/melan-A (Kawakami et al., Proc Natl Acad Sci USA 91(9):3515-3519 (1994), and gp100 (Kawakami et al., Natl Acad Sci USA 91(14):64 (1994)) expressed in normal melanocytes and melanomas, and prostate-specific membrane antigen (PSMA) (Fair et al., Prostate 32(2):140-148 (1997)) and prostate-specific antigen (PSA) (Wang et al., Prostate 2(1):89-96 (1981)), expressed by prostate epithelial cells as well as prostatic carcinomas.

The cell-mediated (T cell) arm of the immune system has been identified as the major immune effector mechanism of tumor rejection. In order for an effective anti-tumor immune response to occur, the recognition of TAA by the T cell is required. This T cell recognition of antigen requires the formation of a complex comprised of: 1) the major histocompatibility complex (MHC); 2) the T-cell receptor (TCR); and 3) a short segment of intracellularly processed antigen enclosed in the MHC molecule.

Upon recognition of antigen associated with the target cell via this process, CD8⁺ T cells (cytotoxic T lymphocytes—CTLs) have the ability to directly kill tumor cells. CD4⁺ T cells (helper T cells, T_{H}) secrete factors, such as interleukin-2 and interferon-□, which support and regulate the functions of CTLs as well as other immune effectors, such as natural killer cells, B cells, and macrophages.

It has more recently been appreciated that the initiation of T cell responses requires a specialized cell type, known as an “antigen-presenting cell” (APC). APCs not only deliver a signal through the binding of the TCR by the antigenic peptide enclosed in the MHC molecule, but also a second co-stimulatory signal to complete the activation sequence. The second signal occurs mainly through CD80/86:CD28, or the CD40:CD40L pathways (Janeway, Cell 76(2):275-285 (1994)). In the absence of these co-stimulatory signals, activation is terminated and the T cell is rendered anergic. Dendritic cells (DCs) are arguably the most efficient APCs known (See, e.g. Steinman, Annu Rev Immunol 9:271-296 (1991)). In their role as a crucial link between antigen, T cell, and the elicitation of an immune response, DCs now occupy the center of an intense investigation into an effective cellular mediator of cancer vaccination.

Dendritic Cells

Dendritic cells (DCs) are bone marrow derived cells that have undergone intense study into their immunostimulatory capacity after their initial recognition as an immune component of lymphoid tissue three decades ago (Steinman and Cohn, J Exp Med 137(5):1142-1162 (1973)). These cells possess cellular characteristics that support their function as very efficient APCs. DCs can uptake and process whole cells or protein, migrate to the lymph nodes, and express high levels of MHC and co-stimulatory molecules required for T cell activation (Banchereau and Steinman, Nature 392(6673):245-252 (1998)). The expression of MHC and co-stimulatory molecules by the dendritic cell—in the context of presentation of antigen—is critical to the engagement and activation of the T cell immunity. Furthermore, they are uniquely able to aggregate T cells at their surface, probably due to their dendritic shape which offers a large area of contact, as well as their high levels of expression of adhesion molecules and integrins (Zhou and Tedder, J Immunol 154(8):3821-3835 (1995); Freudenthal and Steinman, Proc Natl Acad Sci USA 87(19):7698-7702 (1990)). They are the only APCs capable of inducing primary responses in naïve T cells (Steinman, Annu Rev Immunol 9:271-296 (1991)).

Exogenous antigens processed are generally channeled to the MHC class II pathway and transported to the cell surface (Tulp et al., Nature 369(6476):120-126 (1994)). At this point DCs are capable of interaction with CD4⁺ T cells. Antigenic epitopes must be associated with the MHC class I molecules for presentation to cytotoxic CD8⁺ T cells (Jondal et al., Immunity 5(4):295-302 (1996)). Normally, only endogenously synthesized antigens (e.g. those produced in the case of viral infection) are processed via MHC class I pathway. However, leakage or cross-priming between the MHC class I and II pathways allows for presentation of epitopes from exogenous antigens to CD8⁺ T cells (Albert et al., J Exp Med 188(7):1359-1368 (1998); Bennett et al., J Exp Med 186(1):65-70 (1997)). For example, activation of specific CD8+ T cells has been shown following uptake and processing of apoptotic cells by DCs (Albert et al., supra). Maturation of DCs following antigen uptake is characterized by upregulation of adhesion and co-stimulatory molecule expression, as well as redistribution of MHC molecules, resulting in enhanced T cell stimulatory capacity (Banchereau and Steinman, supra).

Dendritic Cell-Based Cancer Vaccines

In generalized terms, DCs operate by engulfing foreign, dying, or otherwise problematic cells and viruses, digesting them, and presenting unique antigenic components of the digested cells to other members of the cell mediated immunity (CMI) via the DC cell surfaces and in the context of the major histocompatibility complex (MHC). It is generally accepted that in this way, DCs make accessible and sensitize other aspects of the immune system (e.g., macrophages, “natural killer” cells, CD8+ cells) to particular target antigens and antigen epitopes, thereby resulting in the clearance from the body of cells bearing these proteins.

Thus, for example, T lymphocytes (i.e., T cells), unlike B lymphocytes (i.e., B cells), generally recognize target antigens only when the antigen is presented in the context of the major histocompatibility complex (MHC). In order to present antigen to T cells, which include T helper cells and cytotoxic T cells, the antigen must be presented in context of an MHC molecule on the surface of an antigen presenting cell. Dendritic cells are perhaps the best antigen presenting cells (APCs), and are thus of keen interest in the area of cancer immunotherapy. In this regard, Steinman, Annu. Rev. Immunol. 9:271-296 (1991) teaches that dendritic cells are rare leukocytes that originate in the bone marrow and can be found distributed throughout the body. Bjork, Clinical Immunology 92:119-127 (1999) teaches that dendritic cells often behave as biological adjuvants in tumor vaccines. Dendritic cells are also known to express several receptors for the Fc portion of immunoglobulin IgG, which mediate the internalization of antigen IgC complexes (ICs). It is generally believed that in this capacity, dendritic cells are used to present tumor antigens to T cells.

Dendritic cell therapy for treating cancer continues to gain interest within the clinical science community. So far, nearly 100 DC cancer vaccine trials have been reported, as summarized, for example, in Ridgway, Cancer Investigation 21(6):873-886 (2003).

At least seven human trials involving prostate cancer patients have been reported in peer-reviewed journals to date. These studies have treated a total of 164 patients with advanced prostate cancer, which include androgen independent cancer, metastatic disease, and biochemical only relapse. The treatments administered 2 to 6 injections of vaccines via intravenous, subcutaneous, intradermal, and intralymphatic routes. The sources of antigen component included peptides, recombinant proteins, and mRNA.

Although results of these studies and the emergence of promising phase III trials that followed highlight the potential of DC-based vaccination as an effective treatment for cancer, such as prostate cancer, there is need and room for further improvement. To date, DC vaccine clinical trials have mainly enrolled patients with progressing androgen-independent prostate cancer (AIPCa), most of whom have been heavily pretreated. These often very ill, immunosuppresed patients with high tumor burden are not good candidates for testing vaccine-based immunotherapy.

Moreover, single antigens do not suffice for effective clearance of tumors, which consist of polyclonal cells and express or lose a whole range of antigens. It is, therefore, important to expose dendritic cells to the proper antigenic profile. Unfortunately, it is often difficult to verify whether this goal has been attained, and there is a risk of leaving out crucial antigenic components, thereby jeopardizing the efficacy of tumor treatment. See, for example, Melero et al., Gene Therapy 7:1167-1170 (2000).

Cryotherapy as a Primary Prostate Cancer Treatment

Practitioners of cryotherapy as a primary prostate cancer treatment believe that cryoablation of prostatic tissue can lead to successful treatment of prostate carcinomas. This long-standing sentiment is based upon the demonstrated destruction induced by freezing temperatures upon living tissue. Several mechanisms of cellular demise following exposure to freezing temperatures have been noted, and include physical (e.g. expansive intracellular ice crystal formation), chemical (protein denaturation), and cellular (e.g. apoptosis) phenomena.

Since no evidence exists to suggest that cancer cells can elude the mechanisms of cryo-induced cellular trauma, the notion persists that successful elimination of carcinomas should result following cryoablation of the prostate.

Cryoablation has been practiced as a treatment for prostate cancer for almost 40 years (Soanes, J Amer Med Asn 196:Suppl. 29 (1966)). In recent years, interest in cryoablation as a primary treatment for other cancers has also emerged, including cancers of the liver (Lee, et al., Radiology 202:624-632 (1997)), kidney (Kam, et al., Journal of Vascular & Interventional Radiology 15:753-758 (2004)), lung (Maiwand, et al., Technology in Cancer Research & Treatment 3:143-150 (2004)), breast (Sabel, et al., Annals of Surgical Oncology 11:542-549 (2004)), and soft tissue sarcomas (Powell, et al., Journal of Urology 158:146-149 (1997)).

It has been demonstrated that an internal tissue temperature of −40 degrees Celsius is required for uniform necrosis of tissue (Larson, et al., Urology 55:547-552 (2000)). At temperatures between −20 and −40 degrees Celsius, cells may encounter osmotic distress (due to extracellular ice formation which results in water withdrawal from the cell), cell membrane rupture, and microthrombi formation (leading to hypoxia). See, Gage, et al., Cryobiology 37:171-186 (1998). Any of these events may lead to lethal (i.e. necrosis, apoptosis) or sub-lethal (i.e. increased cell permeability, alterations in cellular pH) damage to the cell(s). Therefore, cryo-treatment of tissue induces a wide range of fates in tissue, from sub-lethal injury to necrosis.

The most significant complications related to cryoablation of the prostate remain acute urinary obstruction, urinary obstruction requiring transurethral incision of the prostate (TURP), urethral sloughing, and incontinence either primary or secondary to other urinary sequalae.

Thus, despite recent advances in the treatment of cancer, including prostate cancer, there is a need for improved therapies for treating tumors and cancerous tissues. The present invention fulfills this need and provides for further related advantages.

### SUMMARY OF THE INVENTION

The present invention is based, at least in part, on the recognition that the combination of methods capable of liberating tumor antigens with delivery of antigen presenting cells, such as dendritic cells, results in superior tumor treatment. The invention is further based on the finding that, as part of such methods, DCs can be matured in vivo, therefore, DCs not subjected to a separate maturation step ex vivo can be used. The invention is additionally based on recognizing that the administration of antigen presenting cells can be timed such that administration takes place at a time when the bioavailability of the tumor specific antigens is at its maximum. These and other aspects of the invention will be apparent from the disclosure.

In brief; the present invention relates generally to therapeutic methods for treating tumors and cancerous tissues by first distressing cells (e.g., by ablative techniques, including cryoablation, heat ablation, ultrasound therapy, and the like), which results in the liberation of tumor antigens and inflammatory factors, and then delivering one or more selective doses of antigen presenting cells (e.g., autologous DCs) intratumorally or proximate to the tumor or cancerous tissue.

In one aspect, the invention concerns a method for treating a tumor or cancerous tissue in a mammalian subject, comprising

subjecting the tumor or cancerous tissue to cryoablation (or another ablative step, such as, for example, heat ablation or ultrasound therapy), resulting in the liberation of tumor specific antigens;

delivering an effective amount of differentiated antigen presenting cells into or proximate to the tumor or cancerous tissue, whereby at least some of the antigen presenting cells uptake at least some of the tumor specific antigens in vivo; and

allowing an immune response to occur against the tumor or cancerous tissue,

wherein the antigen presenting cells are not subjected to an ex vivo maturation step prior to delivery.

In one embodiment, the ablative treatment, such as cryoablation, results in the release of one or more inflammatory factors, such as, for example, TNF-α and/or IL-1β.

In another embodiment, the released inflammatory factors result in at least partial maturation of the antigen presenting cells in vivo.

In a preferred embodiment, the mammalian subject is a human patient.

Although the described method can be used for the treatment of any tumor, including all types of solid tumors, in a specific embodiment, the tumor is prostate cancer, liver cancer, renal cancer, lung cancer, breast cancer, or soft tissue sarcoma, in particular, prostate cancer.

Cryoablation can be performed following any method known in the art, including total organ cryoablation, typically performed at a temperature of about −40 degrees Celsius, or at about −60 degrees Celsius, and sub-total cryoablation, typically performed at a temperature higher than about −40 degrees Celsius.

The patient treated may have undergone primary cancer therapy prior to cryoablation.

In an embodiment, cryoablation results in necrosis or apoptosis of at least a portion of the tumor cells.

In another embodiment, cryoablation causes sub-lethal damage to at least a portion of the tumor cells.

In a preferred embodiment, the antigen presenting cells are DCs, which were differentiated ex vivo without an additional maturation step. The DCs include autologous DCs of the mammal (human) to be treated, and allogenic DCs.

As noted above, the method of the present invention includes the release of one or more inflammatory factors, such as, for example, TNF-α and/or IL-1β, as a result of tissue cryoablation, which inflammatory factors contribute to at least partial maturation of the antigen presenting (dendritic) cells in vivo.

In a particular embodiment, intratumoral delivery is performed by intratumoral injection of the antigen presenting cells.

In another embodiment, intratumoral delivery is performed through the vasculature of said tumor.

In a further embodiment, the tumor is part of an organ. In this case, intratumoral delivery can be, but does not need to be, performed through direct perfusion of the organ.

In another aspect, the invention concerns a method for treating a tumor or cancerous tissue in a mammalian subject, comprising

subjecting the tumor or cancerous tissue to cellular distress, resulting in the liberation of tumor specific antigen or antigens;

delivering an effective amount of differentiated antigen presenting cells into or proximate to the tumor or cancerous tissue at a time when the bioavailability of the tumor specific antigen or antigens is known or determined to be at about the approximate maximum value, whereby at least some of the antigen presenting cells uptake at least some of the tumor specific antigens in vivo;

and allowing an immune response to occur against the tumor or cancerous tissue.

In a particular embodiment of this method, the antigen presenting cells are not subjected to an ex vivo maturation step prior to delivery.

In another embodiment, the induced cellular distress results in the release of one or more inflammatory factors, such as, for example, TNF-α and/or IL-1β.

Just as in the previous aspect, the preferred mammalian subject is a human patient.

In another embodiment, the tumor is cancer, such as one of the cancers listed above, specifically including prostate cancer.

In a further embodiment, the cellular distress results in lethal injury to at least some of the tumor cells.

In a still further embodiment, the cellular distress results in sub-lethal injury to at least some of the tumor cells.

The cellular injury may, for example, include one or more of necrosis, apoptosis, and osmotic cellular injury.

In another embodiment, cellular distress results from one or more of cryotherapy, heat ablation, chemotherapy, radiation therapy, and ultrasound therapy applied against at least a portion of the tumor or cancerous tissue.

Just as before, preferred antigen presenting cells are DCs, which include autologous DCs of said mammalian subject treated, and allogenic DCs.

In a particular embodiment, the DCs are not subjected to an ex vivo maturation step prior to said delivery.

In another embodiment, the cellular distress results in the release of one or more inflammatory factors, such as, for example, TNF-α and/or IL-1β

Intratumoral delivery may be performed by any method known in the art, including, without limitation, intratumoral injection of the antigen presenting cells, and through the vasculature of the tumor.

When the tumor is part of an organ, intratumoral delivery may also be accomplished through direct perfusion of said organ.

In a further aspect, the invention concerns a therapeutic method for treating a tumor or cancerous tissue residing within an animal having a bloodstream, the method comprising the steps of:

inducing necrosis or apoptosis against at least a portion of the tumor or cancerous tissue by selectively applying cryotherapy, heat ablation, chemotherapy, radiation therapy, ultrasound therapy, or a combination thereof against the tumor or cancerous tissue, thereby liberating cancer-specific antigens from the tumor or cancerous tissue and increasing the bioavailability of the cancer-specific antigens within and proximate to the tumor or cancerous tissue and within in the bloodstream;

monitoring changes in the bioavailability of the cancer-specific antigens in the bloodstream over a period of time;

determining, over the period of time, an approximate maximum value of the bioavailability of the cancer-specific antigens in the bloodstream;

delivering an effective amount of selected antigen presenting cells intratumorally or proximate to the tumor or cancerous tissue when the bioavailability of the cancer-specific antigens in the bloodstream is at about the approximate maximum value and such that at least some of the antigen presenting cells uptake at least some of the cancer-specific antigens in vivo; and

allowing an immune response to occur against the tumor or cancerous tissue.

In a still further embodiment, the invention is directed to a therapeutic method for treating prostate cancer residing within a human body having a bloodstream, the method comprising the steps of:

inducing necrosis or apoptosis against the prostate cancer by selectively freezing at least a portion of the prostate cancer by using cryotherapy, thereby liberating prostate specific antigens from the prostate cancer and increasing the bioavailability of the prostate specific antigens within and proximate to prostate cancer and within in the bloodstream;

monitoring changes in the bio availability of the prostate specific antigens in the bloodstream over a period of time;

determining, over the period of time, an approximate maximum value of the bioavailability of the prostate specific antigens in the bloodstream; and delivering an effective amount of autologous DCs intratumorally or proximate to the prostate cancer when the bioavailability of the prostate specific antigens in the bloodstream is at about the approximate maximum value and such that at least some of the autologous DCs bind to at least some of the prostate specific antigens in vivo; and

allowing an immune response to occur against the prostate cancer.

In yet another aspect, the invention concerns a method for in vivo maturation of DCs, comprising the steps of

subjecting a living tissue to cryoablation; and

administering to the tissue DCs differentiated in the absence of maturation factors.

The tissue can, for example, be a tumor tissue.

In an embodiment, the method further comprises the step of monitoring the in vivo maturation of the DCs.

Monitoring can be performed by any method known in the art, such as, for example, by monitoring the ability of DCs to bind at least one antigen expressed in the tumor tissue.

These and other aspects of the invention disclosed herein will become more evident upon reference to the following detailed description and attached drawings. It is to be understood, however, that various changes, alterations, and substitutions may be made to the specific embodiments disclosed herein without departing from their essential spirit and scope. In addition, it is to be further understood that the drawings are intended to be illustrative and symbolic representations of an exemplary embodiment of the present embodiment and that other non-illustrated embodiments are within the scope of the present invention.

### BRIEF DESCRIPTION OF THE DRAWINGS

FIG. 1 is a typical plot of serum PSA over time following successful brachytherapy of prostate cancer.

FIG. 2 is a typical plot of serum PSA following successful cryotherapy of prostate cancer.

FIG. 3 is a diagram depicting steps involved in the preparation and testing of autologous DCs for intratumoral injection. The timeline for these steps is indicated on the left side of the diagram.

### DETAILED DESCRIPTION OF THE INVENTION

### I. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “antigen presenting cell” (APC) is used in the broadest sense, and refers to specialized type leukocytes, which process complex antigens into smaller fragments by enzymatic degradation, and present them, in association with molecules encoded by MHC, to T cells. Antigen presenting cells specifically include DCs, macrophages, and B-cells, DCs being preferred in the methods of the present invention.

A “dendritic cell” (DC) is an antigen presenting cell (APC) with a characteristic morphology including lamellipodia extending from the dendritic cell body in several directions. Dendritic cells are able to initiate primary, antigen-specific T cell responses both in vitro and in vivo, and direct a strong mixed leukocyte reaction (MLR) compared to peripheral blood leukocytes, splenocytes, B cells and monocytes. DCs can be derived from a number of different hematopoietic precursor cells. For a general description of dendritic cells, including their differentiation and maturation, see, e.g. Steinman, Annu Rev Immunol. 9:271-96 (1991), and Lotze and Thomson, Dendritic Cells, 2nd Edition, Academic Press, 2001.

The terms “cancer specific antigen, and” “tumor specific antigen,” or, briefly, “cancer antigen,” and “tumor antigen,” are used interchangeably, and refer to an antigen that is not present in normal cells (uniquely expressed in cancer/tumor cells) or is differentially expressed in cancer/tumor cells relative to normal cells.

The terms “differentially expressed (antigen),” “differential (antigen) expression” and their synonyms, which are used interchangeably, refer to an antigen whose expression is activated to a higher or lower level in a subject suffering from a disease, specifically cancer, relative to its expression in a normal or control subject. It is understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. For the purpose of this invention, “differential gene expression” is considered to be present when there is at least an about two-fold, preferably at least about four-fold, more preferably at least about six-fold, most preferably at least about ten-fold difference between the expression of a given antigen in normal and tumor (cancer) cells.

The term “tumor,” as used herein, refers to all neoplastic cell growth and proliferation, whether malignant or benign, and all pre-cancerous and cancerous cells and tissues. The term specifically includes cancer and cancerous tissues.

The terms “cancer” and “cancerous” refer to or describe the physiological condition in mammals that is typically characterized by unregulated cell growth. Examples of cancer include but are not limited to, prostate cancer, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The terms “cryoablation,” “cryotherapy,” and “cryosurgery” are used interchangeably and refer to lowering the temperature of a volume of tissue, such as human tumor (cancer) tissue, to sub-freezing temperature in an effort to stress, lethally damage, or inflict sub-lethal injury to the cells in the tissue.

The term “cellular distress” is used to refer to any lethal or sublethal cellular injury that results in an increase in the bioavailability (liberation) of tumor specific antigens. Cellular distress includes, without limitation, necrosis, apoptosis, and osmotic cellular injury, that may result from a variety of treatments, including, for example, cryoablation, chemotherapy, radiation therapy, ultrasound therapy, or any combination thereof applied against at least a portion of the tumor or cancerous tissue.

The terms “tumor specific antigen” and “cancer specific antigen” are used interchangeably and in the broadest sense, including, without limitation, antigens specifically expressed in a certain type of tumor (which are rare), antigens which are differentially expressed in a certain type of tumor, and mutational antigens.

The term “inflammatory factor” is used herein in the broadest sense and includes, without limitation, cytokines, chemokines, and bacterial products involved in inflammation, as well as other molecules that initiate or increase the production of factors involved in inflammation, such as, for example, TNF-α, IL-1α and β, IL-6, and IL-12, macrophage inflammatory proteins 1α and β, and LPS.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®., Bristol-Myers Squibb Oncology, Princeton, N.J.) and docetaxel (TAXOTERE®., Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene(Fareston); and anti-androgens such as flutamide, nilutamide, bicalutamide, leuprolide, and goserelin; and pharmaceutically acceptable salts, acids or derivatives of any of the above.

### II. Detailed Description

The practice of the present invention will employ, unless otherwise indicated, conventional techniques of molecular biology, microbiology, cell biology, and biochemistry, which are within the skill of the art. Such techniques are explained fully in the literature, such as, Cryosurgery for Prostate Cancer Following Radiation Therapy, Erlichman, M. et al. eds., Rockville, Md.: (Springfield, Va.: U.S. Dept. of Health and Human Services, Public Health Service, Agency for Health Care Policy and Research; National Technical Information Service, distributor, 1999); Basics of Cryosurgery Korpan et al., eds., Springer-Verlag, Vienna, 2001; Dendritic Cells: Biology and Clinical Applications, Lotze and Thomson, eds., San Diego, Academic Press, 2001; Dendritic Cell Protocols, Robinson and Stagg, editors, Humana Press, 2005; and Cancer Vaccines and Immunotherapy Stern, Peter et al., Cambridge University Press, 2000.

The present invention is based, at least in part, on the recognition that what was observed in the early reported remissions of metastatic disease following local treatment of prostate cancer was in fact, at least partially, due to the creation of cancer antigen bioavailability, that was followed by a systemic cell mediated response.

The present invention is further based on the recognition that the various non-surgical treatments for cancer (especially radiation therapy but also cryotherapy, chemotherapy, and ultrasound therapy) result in the demise of many of the tumor cells these treatments target, but that the ultimate “clearance” of cancer following treatment is due to the role of the immune system. This recognition is based primarily on the “systemic” view of cancer; that is, that cancer cells have metastasized—or spread from the primary cancer to other parts of the body by the time a diagnosis of cancer is made. This view holds that the “classical” view of cancer—that cancer can be diagnosed while confined to a particular organ—is based only on our inability to image or otherwise detect nests of occult malignancy at the cellular level.

Indeed, the “systemic” view has become more mainstream as evidence has mounted that cells from tumors that are thought to be localized to the originating organ (largely known as Ti and T2 cancers) have in fact migrated elsewhere by the time of diagnosis. For instance, a recent report demonstrates that prostate cancer cells can be isolated from bone marrow in 50% of “localized” prostate cancers. Ellis W J, Pfitzenmaier J, Cclii J, Arfman E, Lange P H, Vessella R I, Journal of Urology 61(2):277-281 (2003). If a significant number of “localized” cancers have in fact migrated beyond their site of origin by the time of diagnosis, then the high control rates seen following locally directed treatment for localized disease—approximately 90% at 5 years following brachytherapy or focused radiation for prostate cancer—might be due to the clearance of these cells by the immune system perhaps aided by the primary local treatment. This hypothesis is consistent with a recently observed phenomenon that follows brachytherapy of prostate cancer, which is discussed below.

An important tool in the diagnosis of prostate cancer, and in monitoring the efficacy of treatment and potential cancer recurrence, is the Prostate Specific Antigen (PSA), which is a 33 kDa human kallikrein-family serine protease produced exclusively by glandular components of the human prostate. Serum levels in young males are generally undetectable; with age however, men are found to have circulating levels between 0.5 and 4.0 ng/ml benignly, but above 4.0 ng/ml are found to be of greater risk of harboring cancer of the prostate. Importantly, PSA is produced in males of reproductive age but is confined to prostatic cells and the ducts which connect the prostate to the prostatic urethra. An enzyme, PSA is thought to play a role in the liquefaction of the ejaculate, the putative primary role of the prostate gland. As noted above, serum PSA has value as both a marker for prostate cancer when sufficiently elevated, and as a means of tracking the success of therapy after definitive treatment for carcinoma of the prostate. Following successful curative therapy, serum PSA should approach non-detectable levels.

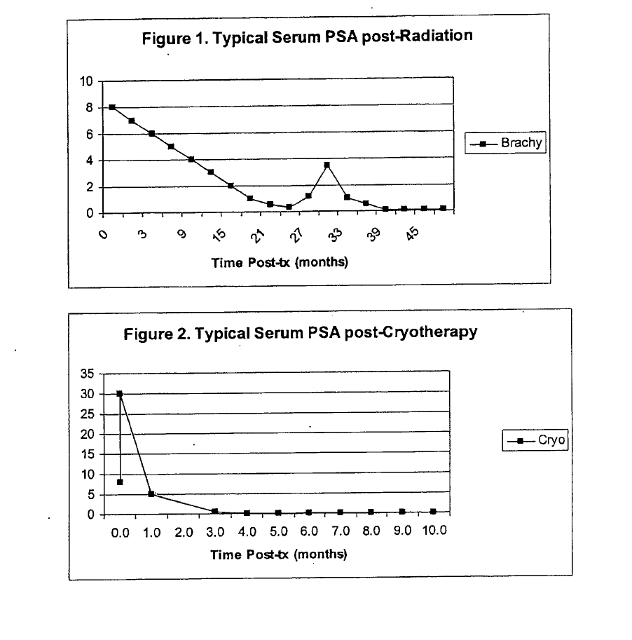

In brachytherapy, which is a common treatment for prostate cancer, rice-sized titanium pellets coated with radionuclide are implanted into the prostate in order to deliver a tumoricidal dose of radiation to the prostate, so as not to deliver radiation anywhere beyond the prostate itself A typical plot of serum PSA over time following successful brachytherapy of prostate cancer is depicted in FIG. 1. The “spike” in PSA observed at 28 months in FIG. 1 is typical of what has been termed the PSA “bounce.” Merrick C S, Butler W M, Wallner K E, Galbreath R W, Anderson R L, Journal of Radiation Oncology, Biology, Physics 54(2):450-456 (2002); Cavanagh W, Blask J C, Grimm P D, Sylvester I F, Seminars in Urologic Oncology 18(2):160-165 (2000). While no definitive explanation currently accounts for this observation, it is known that—following radiation—cells with intact, wild-type p53 are able to maintain the G(2) arrest cycle for a prolonged time interval following ionizing radiation. Scott S L, Earler J D, Gumerlock P H, Cancer Research 63(21):7190-7196 (2003). However long this cycle can be maintained in the presence of severe DNA damage from ionizing radiation, though, eventually the cells must enter M phase. At that point a certain number of cells, cancerous and not, will perish, mainly through the apoptotic pathway, secondary to the severe DNA damage (double strand breaks) incurred by the radiation. Putatively, such a clonogenic demise will result in an increase in serum PSA in a transient fashion.

Regardless of whether one accepts this underlying mechanism as the cause of the serum PSA bounce, it is clear that it is observed in a large proportion of prostate cancer patients treated with radiation, typically between 18 and 36 months.

Without being bound by any particular theory, looking at the totality of evidence it is reasonable to conclude that the release of cellular contents at some time following radiation treatment allows for the bioavailability of cancer-specific antigen(s)/protein(s), and that uptake of this material—by DCs or other aspects of the cell-mediated immunity—provides the potential for a systemic immune response.

Although PSA is used to measure peak antigen availability, PSA is acting merely as an index for this event. In fact, unknown numbers of additional discrete protein entities are released concurrently. These entities may be cancer-specific antigens, may be proteins over-expressed by cancer cells, or may be expressed by both benign and cancer cells. In any of these three events, the availability of these putative protein entities, if processed by cells such as DCs, may lead to a systemic anti-metastatic immune response.

Two recent animal studies bear mention at this point: First, Teitz Tennenbaum, et al., Cancer Research 63:8466-8475 (2003) report on the statistically significant improvement in anti-tumor efficacy in a murine model with two different tumor lines using DC therapy and radiation. Most notably, the anti-tumor effect was potentiated with the use of both modalities together, namely, the effect on the combined treatment group was greater than the additive efficacies noted in the arms treatment via autologous DCs only and radiation only. Secondly, three reports describe the disappearance of transplanted tumor in a mouse model following administration of systemic chemotherapy followed by intratumoral injection of DCs (Tong, et al., Cancer Research 61:7530-7535 (2001); Shin, et al., Histology & Histopathology 18:435-447 (2003), and Yu, et al., Clinical Cancer Research 9:285-294 (2003)). Interestingly, when tumors were implanted on both right and left flanks with DCs injected into one side only, the contralateral tumor was noted to regress. Both studies suggest that damage to cells in the tumor by radiation or chemotherapy, followed by the local introduction of DCs results in a more effective clearance of tumor that would have been expected, again suggesting a systemic, immune-mediated effect. Accordingly, if one assumes that evidence exists supporting the notion of a systemic immune response to cancer based on the treatment of the primary tumor and that the basis for assuming this rests with some type of damage or disintegration of the primary tumor and the subsequent bioavailability of cancer related antigen, we can then conclude that there might exist more optimal means of liberating antigen for maximum bioavailability to antigen presenting cells, as would be the case following ablative therapy.

FIG. 2 depicts the typical PSA pattern following successful cryotherapy: a large magnitude spike within hours/days of treatment—as a result of large scale necrosis of the prostate mass—followed by non-detectable serum levels shortly thereafter. Wieder J, Schmidt J D, Casola G, vanSonnenberg E, Stainken B F, Parsons C L Journal of Urology 154(2 Pt 1):435-441 (1985). If one is to use serum PSA, as mentioned above, as an indicator or index of a large availability of antigen for immune processing and response, it may be concluded that following cryotherapy the index is—compared to radiation treatment—(1) of greater magnitude, and (2) of less variance in timing and duration. Furthermore, it is proposed that an even more optimized and specific program of timing the interval between primary treatment (cryotreatment in this case) and DC treatment exists.

Thus, and more generally, therapeutic methods including the steps of first inducing cellular distress (including lethal and sub-lethal cellular injuries, such as, for example, necrosis, apoptosis, osmotic cellular injury, and the like) by any means (e.g., cryoablation, chemotherapy, radiation therapy, ultrasound therapy, or any combination thereof applied against at least a portion of the tumor or cancerous tissue), and then delivering one or more selective doses of antigen presenting cells (e.g., autologous DCs) intratumorally or proximate to the tumor or cancerous tissue, provide a new and advanced approach to tumor/cancer treatment.

In the method of the present invention, preferably DCs not subjected to a maturation step ex vivo are delivered to a tumor or cancerous tissue. Immature DCs take up and process tumor antigens made available by cellular distress, such as cryotherapy. A known disadvantage of using immature DCs is, however, that they are less efficient than mature DCs in their ability to migrate to the lymph node, and to activate T cells. Surprisingly, the methods of the present invention allow the use of immature DCs without compromising efficiency of T cell activation. Without being bound by any theory, a likely explanation for this result is that the DCs mature after delivery, due to the presence of inflammatory factors released by the treatment resulting in cellular distress, such as cryotherapy. The present invention also provides a method for in vivo maturation of DCs, taking advantage of this phenomenon.

In a particular embodiment, antigen presenting cells are delivered after a selected period of time sufficient for the bioavailability of liberated cancer-specific antigens (if needed, monitored over the selected period of time) resulting from the cellular distress to be at or near a maximum value. However, as discussed below in more detail, it is not always necessary to monitor the bioavailability of tumor antigens or to delay the delivery of antigen presenting cells.

As noted above, the present invention relates generally to immunotherapy and, more specifically, to methods for treating tumors and cancerous tissues by delivering antigen presenting cells such as, for example, DCs, which had not been exposed to maturation factors ex vivo, intratumorally or proximate to the tumor or cancerous tissue, preferably when the bioavailability of cancer-specific antigens is at or near a maximum value. Thus, the invention provides a new type of APC-based approach to the treatment of tumors and cancers. The in situ availability of cancer antigens may be accomplished in any one of several ways such as, for example, by selectively applying cryoablation, chemotherapy, radiation therapy, ultrasound therapy, or any combination thereof, against the tumor or cancerous tissue as is known in the art.

Known cancer treatments, including, for example, radiation therapy, chemotherapy, ultrasound therapy, and cryoablation therapy, result in lethal or sublethal damage in tumor cells, typically leaving mostly necrotic or apoptotic cells and minimal remaining viable neoplastic cells in the tumor tissue, and lead to the liberation of cancer antigens. An important recognition underlying the present invention is that these effects open a great window of opportunity for an effective immunotherapeutic strategy using injection of APCs, such as DCs, following these standard therapies. Particularly suitable for this approach is a combination of cryoablation and APC-based immunotherapy. Unlike other conventional modalities for cancer, such as, prostate cancer, cryoablation leads to immediate liberation of antigen, does not compromise the immune system, and can be repeated without fear of excessive toxicity. In addition, antigen liberation induced by cryoablation is not only immediate but also more concentrated, i.e. occurs within a narrower time frame. For all these reasons, cryoablation represents the most appealing primary treatment of choice to precede APC-based immunotherapy, however, other primary cancer treatments followed by the injection of APCs, such as DCs, are also part of the invention.

Total organ cryoablation, with its attendant complications, is not required for this combination therapy. “Sub-total” cryoablation of the cancerous organ, such as the prostate, is believed to be sufficient to liberate tumor-associated antigen and provide apoptotic/necrotic cells for uptake by injected APCs, such as DCs. Sub-total cryoablation may be defined as either 1) the ablation of less than 100% of the organ, for instance the prostate, or 2) cryotreatment of the tissue to greater than cryoablation temperatures, i.e. greater than −40 degrees Celsius. In definition 1), the sub-total descriptor refers to a volumetric context, while if definition 2) is used, “sub-total” is referenced in a temperature context. In either event, the cryoablation is not total ablation, which may be defined as cryotreatment sufficient to induce uniform and confluent necrosis of a tissue or organ in its entirety. Total cryoablation is typically achieved by freezing the whole volume of the tissue or organ to −40 degrees Celsius for a period of three minutes, or a temperature of −60 degrees Celsius for one minute (Larson, et al., Urology 55(4):547-552 (2000)).

In particular, the present invention provides an alternative strategy to the ex vivo loading of target antigen to enriched autologous DCs. A problem associated with the ex vivo loading of target antigen to enriched autologous DCs relates to the lack of certainty as to whether the DCs have been exposed to the proper antigenic profile. As discussed earlier, it is often difficult to assure and verify that the DCs are exposed to all crucial antigenic components, which may compromise the success of tumor treatment.

According to the present invention, one or more cancer antigen(s) is/are liberated in vivo, followed by the application of a large volume of autologous DCs directly to the location or near the location of the liberated antigen or antigens. It is believed that in this way, the DCs, by causing macropinocytosis or, in some instances, endocytosis, of soluble proteins liberated from cancer cells and by phagocytosing the remnants of dead/dying cells (including the cellular membrane of these cells), and then migrating to the lymph nodes to contact the cell mediated and humoral aspects of the immune system, will lead to a systemic response against the cancer.

In one embodiment of the present invention, liberation of the target antigen(s) (e.g. by cryoablation) is followed by direct, intratumoral (IT) injection of non-loaded DCs, which have not been subjected to a prior ex vivo maturation step. Upon injection, DCs take up antigen from apoptotic or necrotic tumor cells within the tumor bed. Since the tumor cells are the source of antigen in vivo, IT injection foregoes the need for the selection, costly manufacturing under GIMP conditions, and in vitro loading of tumor antigens. Since cryoablation or other treatment of tumor cells also releases certain inflammatory factors, such as TNF-α and IL-1β, the DCs undergo in vivo maturation, which enhances their ability to migrate to the lymph node and activate tumor-specific T cells in the lymph node. As a result, the methods of the present invention represent a significant advance in the immunotherapy of cancer.

Cryoablation in combination with intratumoral APC injection may be useful for any cancer patient for whom tumors or cancerous tissue may be detected or imaged using available diagnostic methods. For those patients for whom no visible tumor can be detected or imaged by available diagnostic methods, including patients who experience presumptive tumor recurrence after undergoing primary therapies, the combination of “sub-total” cryoablation (as discussed above) and direct injection of PACs may still be appropriate, if the area thus treated is either proximate to the former location of the tumor, or within the organ or tissue previously known to be cancerous.

The rationale for this strategy is as follows:
a) residual cancer cells or pre-malignant cells may be present in the organ, e.g. prostate to serve as antigen source for injected DCs
b) various tissue-specific antigens are expressed by both cancer and normal cells of the affected tissue or organ (e.g., PSA, PSMA, PAP etc. for prostate); anti-tumor immune responses can be induced by this procedure directed towards these shared antigens. Cross-reactivity against normal tissues is anticipated and is within this scenario regarded as an acceptable effect.

Both local and systemic immune responses are theoretically generated using this procedure, thus allowing for the elimination of not just cancer cells within the organ or tissue, e.g. prostate, but also metastatic lesions in other parts of the body.

Primary treatments, such as radiation therapy, chemotherapy and cryoablation, are performed following known protocols. A particular protocol for cryoablation, as part of the treatment of prostate cancer, is provided in the Examples below. Thus, cryoablation can, for example, be performed using the commercially available Endocare Cryocare CS® system (Endocare, Inc.). The CS system uses liquid argon gas as a cryogen, and liquid helium as a warming agent. Through thermocouple feedback, this system allows for controlled freezing of the volume of the tissue targeted, and “active” thawing of the same volume, either at the discretion of the physician or automatically via use of a computer-mediated system. In addition, the Cryocare CS® system employs integrated ultrasound, which allows the operator to monitor all aspects of planning, probe placement and the progress of the freezing event via ultrasound on one unit.

Production and testing of autologous DCs for use in the methods of the present invention can also be performed following techniques known in the art. Lacking known specific cell markers, DCs can, for example, be purified by removal of other defined cell populations, such as T and B lymphocytes, natural killer cells and monocytes, by using antibodies and magnetic beads, panning or a cell sorter (Banchereau and Steinman, Nature 392:245 (1998); Freundenthal and Steinman, Proc. Natl. Acad. Sci. USA 87:7698 (1990); Steinman, Annu. Rev. Immunol. 9:271 (1991)). However, DCs are known to be present in low abundance in accessible biological samples, such as blood. Discovery of methods differentiating DCs from their precursors allows for much larger yields, as a result of removing other lymphocytic components.

Monocytes, which are among the most abundant DC precursors in blood, can be differentiated into DCs in vitro typically using a combination of cytokines, most frequently granulocyte macrophage-colony stimulating factor (GM-CSF) in combination with one or more additional cytokines, such as, for example, one or more of interleukin-4 (IL-4) interleukin-7 (IL-7), interleukin-13 (IL-13) and IFN-α. Methods for in vitro differentiation of monocytes into DCs in a medium including GM-CSF, IL-4 and TNF-α are described in U.S. Pat. No. 5,849,589. The use of IL-7 to induce monocyte differentiation and DC maturation has been described, for example, by Fry and Mackall, Blood 99:3892-3904 (2002); Li, et al., Scand. J. Immunol. 51:361-371 (2000), and Takahashi, et al., Human Immunol. 55:103-116 (1997). According to U.S. Pat. Nos. 6,524,855 and 6,607,722, monocyte differentiation is initiated by subjecting the monocytes to photophoresis by exposure to a photoactivatable agent which is capable of forming photo-adducts with cellular components, and then irradiating the exposed cells with radiation suitable for activating the agent, typically ultraviolet or visible light.

In a particular embodiment, differentiation is performed in the presence of GM-CSF and IFN-α. Although there is a divide in the literature about the putative functional benefit of DCs cultured in GM-CSF and IFN-α, it is believed that this system offers several advantages. Such benefits may include short term cultivation, higher expression of molecules involved in antigen presentation, appearance of at least partially mature phenotype in a significant portion of cells (without adding additional maturation factors), and efficient stimulation of humoral and cellular arm of immune response (see, e.g. Santini et al., Stem Cells 21:357-362 (2003)).

DC precursors may be isolated by a variety of methods known in the art, including plating, separation on magnetic beads (e.g. Dynabeads®, Dynal Biotech, Oslo, Norway), tangential gel filtration, or using the Elutra Cell Separation System (Gambro BCT, Lakewood, Colo., USA). Certain methods known in the art for in vitro DC generation from monocytes involves adhesion of these DC precursors to tissue culture plastic, followed by removal of non-adherent cells, and a period of culture in the presence of appropriate cytokines. Since this process is labor intensive, and has the potential for contamination due to an open culture system, monocyte isolation and DC culture can also be conducted in a closed system, such as, for example, in cell factories or culture bags (Beger et al., J. Immunol. Methods 268:131 (2002); Guyre et al., J. Immunol. Methods 262:85 (2002)). Using improved methods known in the art and commercially available equipment, a population of cells comprising up to about 80% immature DCs can be generated.

Most methods rely on the in vitro development of DC-like cells from CD34⁺ progenitor cells or blood monocytes (see, e.g., Caux, et al., Nature 360:258 (1992); Romani, et al., J. Exp. Med. 180:83 (1994); Sallusto et al., J. Exp. Med. 179:1109 (1994)). According to these methods, monocytes are usually cultured for 5-7 days with GM-CSF and IL-4 to generate immature DCs that are subsequently activated to obtain mature DCs with full T stimulatory capacity. Type I interferons have also been described to induce rapid differentiation of monocytes into DCs. (Santini, et al., J. Exp. Med. 191:1777-1788 (2000)).

Various factors discovered for maturing DCs in vitro (ex vivo) include monocyte-conditioned media (MCM), TNF-α, and/or other maturation factors, such as LPS, IL1-β, and bacillus calmette guerrin (BCG), optionally in combination with other factors like prostaglandin-E2 (PGE2), vasoactive intestinal peptide, poly-dIdC, as well as mycobacterial cell wall components.

It is generally accepted that the degree of maturity of DCs is an important consideration in the generation of an effective cancer vaccine (Onaitis et al., Surg. Oncol. Clin N. Am. 11(3):645-660 (2002)). Defective dendritic cell function due to the accumulation of immature DCs has been implicated as a mechanism of immune suppression in cancer (Almand et al., J. Immunol. 166(1):678-698 (2001)). Maturing DCs undergo changes that result in augmentation of their capacity to activate T cells as they increase antigen density on the surface, as well as the magnitude of the T cell activation signal through the co-stimulatory molecules (Zhou and Tedder, Proc. Natl. Acad. Sci. USA 93(6):2588-2592 (1996)). In addition, maturing DCs develop the capacity to migrate to the lymph nodes, where T cell activation generally occurs (Banchereau and Steinman, Nature 392(6673):245-252 (1998)).

Mature DCs, however, also lose their capacity to uptake and process antigens. For that reason, according to the present invention, DCs are not subjected to a separate maturation step, in the presence of maturation factors. In other words, the methods of the present invention use DCs from monocytes, which are obtained by culturing monocytes in the presence of differentiation factors, without additional incubation in the presence of maturation factors (e.g., monocyte conditioned media, LPS, TNF-α, IL1-β and bacillus calmette guerrin (BCG)). Without being bound by any particular theory or mechanism, it is believed that DCs not subjected to a separate maturation step can be successfully used in the methods of the present invention since cryotherapy results in the release of inflammatory factors that, directly or indirectly, induce DC maturation in vivo.

Another important DC characteristic is the ability to secrete biologically active IL-12 when DCs are in the process of activating naïve T cells. IL-12 is a cytokine that induces a Th1 type response (Kennedy et. al., Eur. J. Immun 24 (10):2271-2279 (1994). This type of T cell response results in the induction and differentiation of cytotoxic T lymphocytes (CTL), which constitute the effector arm of the immune system most effective in combating tumor growth. IL-12 also induces growth of natural killer (NK) cells (Kobayashi et. al., J. Exp. Med. 170(3):827-845 (1989)) and has anti-angiogenic activity (Voest et. al., J Natl, Cancer Inst. 87(8):581-586 (1995)), both of which are effective anti-tumor weapons. The use of DCs that produce IL-12 is therefore, in theory, optimally suited for use in DC-based cancer therapy.

Snijders et. al. was the first group to report that exposure to interferon-γ (IFN-γ) is essential in DC ability to secrete IL-12 during engagement with T cells through the CD40-CD40 ligand interaction (Snijders et. al., Int. Immunol. 10(11):1593-1598 (1998)). The same group also reported that exposure to IFN-γ before, during or slightly after the process of DC maturation is important in DCs' ability to produce IL-12 (Vieira et. al., J. Immunol. 184:4507-4512 (2000)). In contrast to the profound modulation of the IL-12-producing capacity, IFN-γ did not affect the maturation-associated phenotypical changes, neither elevating nor inhibiting the expression of the mature DC marker CD83, the costimulatory molecules CD40, CD80, and CD86, and the class II MHC Ag-presenting molecule HLA-DR (Vieira et. al., J. Immunol. 184:4507-4512 (2000)). In order to take advantage of the beneficial properties of IFN-γ, in a preferred embodiment, the differentiated DCs of the present invention are exposed to IFN-γ after culture.

A particular protocol of DC preparation according to the present invention involves the following steps: (1) leukopheresis of patients, (2) isolation of DC precursors (monocytes), (3) culture and differentiation of DCs, without a separate maturation step (optionally followed by IFN-γ treatment), and (4) harvest and cryopreservation of autologous DCs. Particular protocols for performing these steps are provided in the Examples below, however, other protocols known in the art, including modifications and adaptations to a particular task, are also suitable for performing the methods of the present invention, and are within the scope herein.

Leukapheresis starts with the separation of whole blood into red blood cells (RBCs), polymorphonuclear (PMN) cells, mononuclear cells, and the platelet-rich plasma. Thereafter, the mononuclear cells are collected, and the PMN and RBCs are mixed with the platelet-rich plasma and returned to the patient. This is followed by the isolation of DC precursors (monocytes), using a commercial equipment, such as, for example, the ELUTRA™ cell separation system (Gambro), culture and differentiation of DCs, and harvest and preservation of immature autologous DCs.

According to the present invention, instead of in vitro loading of DCs with a tumor-associated antigen (TAA) for the purposes of vaccination, a direct, intratumoral (IT) injection of non-loaded DCs is used. Upon injection, DCs theoretically take up antigen from apoptotic or necrotic tumor cells within the tumor bed. Since the tumor cells are the source of antigen in vivo, IT injection foregoes the need for the selection, costly manufacturing under GMP conditions, and in vitro loading of tumor antigens. Intratumoral injection of DCs has been tested in human clinical trials; one study demonstrated tumor regression in 4 of 7 patients with metastatic melanoma and 2 of 3 patients with breast carcinoma (Triozzi et al., Cancer 89(12): 2646-2654 (2000)). Biopsies of the regressing lesions demonstrated infiltrating T cells, suggesting that injected DCs had indeed activated an immune response against the tumor cells. A particular protocol for IT injections of DCs is described in the Examples below.

Although in some embodiments direct intratumoral injection may be preferred, other methods of intratumoral delivery are also known and suitable for practicing the present invention. Such methods include, for example, delivery of the antigen-presenting cells through the vasculature of the tumor. Alternatively, the cancerous organ can be perfused in a solution comprising the antigen presenting cells, e.g. DC's. All these and similar embodiments are specifically within the scope of the invention.

An important aspect of certain aspects of the invention is the timing of DC administration. After inducing necrosis or apoptosis, for example by cryotherapy, chemotherapy, radiation therapy, ultrasound therapy, or a combination thereof, DCs are administered after allowing sufficient time for the liberation of tumor antigens. An effective amount of selected antigen presenting cells (e.g. DCs) are delivered intratumorally or proximate to the tumor or cancerous tissue when the bioavailability of the cancer-specific antigens in the bloodstream is at about the approximate maximum value and such that at least some of the antigen presenting cells (e.g. DCs) uptake at least some of the cancer-specific antigens in vivo.

If necessary, the bioavailability of antigens can be monitored using any assay format suitable for detecting a particular tumor-associate antigen or a group of antigens. Suitable methods of antigen detection include, without limitation, immunoassays, which may be in ELISA format, antibody-based chemoluminescence assays, and assays measuring a bioactivity of the tumor antigen. Methods for detection PSA levels are well known in the art, including immunometric assays using an antibody-coated bead to capture PSA in the test sample and enzyme labeled antibody to generate a signal which is read chemiluminescently. Several PSA assays are commercially available, such as, for example, the IMMULITE and IMMULITE 2000 Third Generation PSA Assays (Diagnostic Products Corp., DPC); Tandem-E PSA/Tandem-R free PSA assay (Hybritech).

Other known prostate tumor antigens include prostatic acid phosphatase (PAP) and prostate specific membrane antigen (PSMA), which can be detected using similar assays. Carcinoembryonic antigen (CEA) is known to be associated with cancers of the gastrointestinal tract. Breast, lung, and other solid cancers also have known markers, or markers that can be readily identified by standard methods of gene expression or proteomic analysis. The detection of such markers circulating in the blood stream can be performed by methods known in the art, such as those discussed above. Indeed, cryoablation might increase the number of such markers, releasing additional tumor antigens that do not normally circulate into the system. Accordingly, virtually any tumor antigen, or any combination of tumor antigens, can be used to monitor the liberation of antigen, when such monitoring is needed as part of the present invention.

Further details of the invention are illustrated by the following non-limiting Examples.

### Example 1

### Production and Testing of Autologous Dendritic Cells

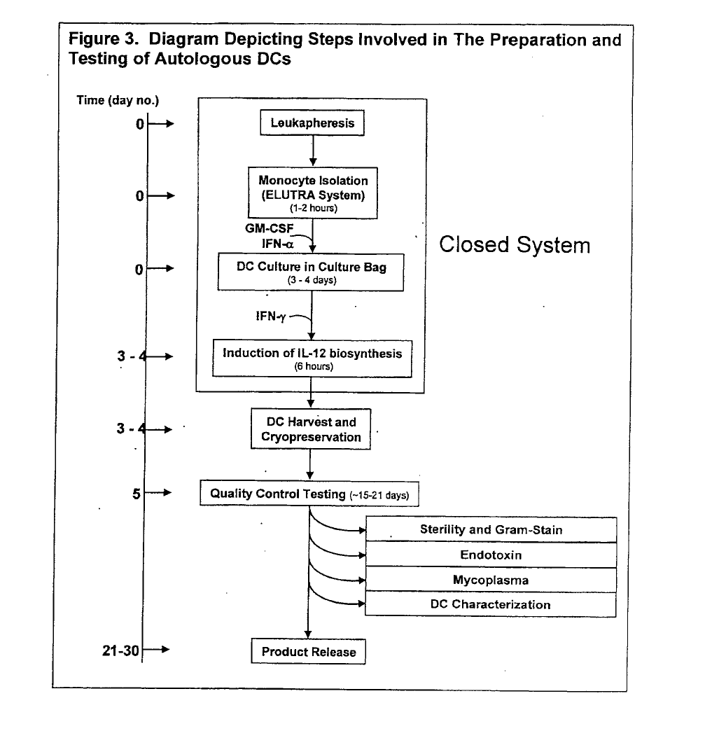

FIG. 3 is a flow diagram, illustrating the steps of the preparation and testing of autologous dendritic cells (DCs).

The production process of autologous DCs can be divided into 4 steps: (1) leukopheresis of patients, (2) isolation of DC (monocytes) using the Gambro ELUTRA™ system, (3) culture and maturation of DCs in a gas permeable bag, (4) harvest and cryopreservation of autologous DCs. Each of these steps is described below.

1. Leukapheresis of Patients

A single-stage White Blood Cell (WBC) Channel (or chamber) is used to collect the mononuclear cells. The anticoagulated whole blood enters the chamber through the inlet tubing. As it flows into the channel, it is separated into 3 blood components, the red blood cells (RBC), the WBCs, and the platelet-rich plasma. The separation of all 3 of these components is controlled by the specific gravity differences between the blood components and the pressure, density, and viscosity flowing through the tubing. The individual components are drawn from the separation chamber through dedicated tubing and collected in the respective receiving bags. In addition, leukapheresis also separates the majority of polymorphonuclear neutrophils (PMNs) from the mononuclear cells. In the end, the mononuclear cells are collected while the RBCs are mixed with the platelet-rich plasma and returned to the patient.

During processing, the separation and collection is monitored by a number of optical and ultrasonic sensors. The sensors are capable of detecting conditions such as low anticoagulant levels, inlet air, detection of RBCs at key locations, platelet concentration, etc.

2. Isolation of DC Precursors (Monocytes) Using The Gambro ELUTRA™ System

The leukapheresis material is processed for the isolation of dendritic cell precursors (monocytes) using the Gambro ELUTRA™ System. The ELUTRA™ System is a semi-automatic, centrifuge-based laboratory equipment that uses counter-flow elutriation technology to separate cell products, such as leukapheresis products, into multiple fractions based on cell size and specific gravity. It utilizes a sterile disposable set, which incorporates separation chamber and product collection bags. Thus, unlike conventional elutriation systems, the ELUTRA™ is a closed cell separation system that does not require dismantling and sterilization of the separation chamber and rotor after each run.

This system comes with 9 different elutriation profiles, including a pre-programmed profile for monocyte enrichment. Rouard et al., (Transfusion 43(4):481-487 (2003)) has previously reported preliminary studies that lead to the invention of the ELUTRA™ System for monocyte isolation. This system reproducibly provides products with >80% monocyte purity and >60% monocyte recovery from a typical leukapheresis product in one hour.

Prior to the start of the monocyte enrichment process, 5 mL of the leukapheresis material is sampled and sent for hematological analyses. Information on red blood cell (RBC) and white blood cell (WBC) concentrations within the leukapheresis material is essential for the initiation process of the ELUTRA™ System. In cases where the leukapheresis materials contain excess RBC, the system provides an optional RBC debulking step to assure proper monocyte enrichment procedure.

Prior to loading onto to the system, the disposable tubing set is connected to media and collection bags using a sterile connect device. The front panel of the ELUTRA™ System shows the system flow path to aid the operator in loading the disposable tubing set. After the tubing set is loaded, the system loads the pumps, performs a fluid leak detection, and prime the tubing set by replacing the air within with elutriation media (Hanks Balance Salt Solution Cambrex, Walkersville, Md.) and 1% human serum albumin (HSA; Plasbumin®, BayerAG, Leverkusen, Germany).

If the RBC debulking step is recommended, the system loads cells from the starting cell product bag into the separation chamber and allows the cells to sediment. RBCs are removed from the bottom of the separation chamber. This step takes approximately one hour. After the debulking has been completed, the system pumps media into the cell bed and adjust the flow rates and/or centrifuge speed, and proceed to the elutriation step. This step, which also takes approximately one hour, collects a total of 5 cell fractions. At the conclusion of the elutriation step, the system prompts the operator to seal all collection bags and disconnect them from the tubing set, followed by additional prompts to remove the elutriation chamber, unload the pumps and remove the rest of the tubing set for disposal.

The first 4 fractions contain mainly platelets, RBC, and lymphocytes. These fractions are discarded. The fifth fraction contains the enriched monocyte population to be used as precursor cells for DC production. For this fraction, cells are collected using media compatible with DC culture (Dulbecco Modified Eagle Media (DMEM; Cambrex, Walkersville, Md.) containing 2% HSA (Plasbumin®, BayerAG, Leverkusen, Germany). If RBC debulking is not recommended, the System proceeds immediately to the elutriation step as described. 3. Culturing Monocytes

Monocytes are cultured and differentiated by any method known in the art, such as those discussed above. Such methods are also disclosed in standard textbooks, such as, for example, Dendritic Cell Protocols, Robinson and Stagg, editors, Humana Press, 2005. In a typical protocol, monocytes are cultures in the presence of GM-CSF in the presence of one or more additional cytokines (e.g., IL-4, IL-7, IL-13 and/or IFN-α), without additional incubation in the presence of maturation factors, such as, for example, LPS, TNF-α, IL1-β). In a particular embodiment, a collection bag, containing a monocyte fraction from the ELUTRA™ system is connected to a gas permeable culture gab (e.g. Permalife™, Origen Biomedical, Austin, Tex.), where the cell suspension is transferred into the culture bag by gravity. GM-CSF and IFN-α are added to the culture bag, which is then incubated, typically at 37° C. and in the presence of 5% CO₂ for 3-4 days. Prior to harvest, IFN-γ may be added to the culture to promote IL-12 biosynthesis during DC interaction with T cells.

4. Harvest and Cryopreservation of Autologous DCs

Cell suspensions are transferred into a 250 mL centrifuge tube, and centrifuged for 10 minutes at 1200 rpm. Culture supernatant is removed and each cell pellet is resuspended in 10 m PBS. Cell suspensions are pooled into two 50 mL conical centrifuge tubes (2×10 mL each). The four 250 mL centrifuge tubes are rinsed with 10 mL PBS; the rinse is pooled with the DC suspension. The two 50 mL tubes are centrifuged for 10 minutes at 1200 rpm (wash 1). Supernatant is removed and each cell pellet is resuspended in 10 mL PBS. Thirty mL of PBS is added into each tube prior to another centrifugation for 10 minutes at 1200 rpm (wash 2). Supernatant is removed and each cell pellet is resuspended in 10 mL PBS. Cell suspension is pooled and the volume is adjusted to 40 mL. Cell count is performed using a hemocytometer. Trypan blue is used to visualize dead cells. The number of live DCs is approximated using the number of large, trypan blue-negative cells. The cell suspension in the 50 mL tube is centrifuged for 10 minutes at 1200 rpm (wash 3). Supernatant is removed and the cell pellet is resuspended in the appropriate volume of cryopreservation media (6% Pentastarch, Baxter, Deerfield, Ill.), 4% USP human serum albumin (Plasbumin®, BayerAG, Leverkusen, Germany), 5% dimethyl sulfoxide (DMSO, Sigma, St. Louis, Mo.) to achieve a concentration of 14-20×10⁶ live DCs/mL. One half mL of cell suspension is transferred into each cryovial, representing 7-10×10⁶ DCs/vial. Each vial will be labeled with a product number, a lot number, date of harvest and expiration date. These vials are immediately transferred into a −80° C. freezer. After 12 hours, these vials are transferred into a liquid Nitrogen freezer. At least 10 vials are cryopreserved. Four vials are dedicated for injection, four vials for quality control testing, and the remaining vials are kept for product retention.

DCs are then subject to quality tests known in the art, such as, for example, sterility tests (fungi, gram positive and negative bacteria), endotoxin and gram-stain tests, mycoplasma test and DC characterization (cell count, viability and purity).

### Example 2

### Cryoablation and Intraprostatic DC Injection

The rationale for this protocol is based on the recognition that the liberation of tumor-associated antigen or prostate-associated antigen resultant from the cryoablation event allows the locally injected, autologous dendritic cells to uptake antigen, migrate to the lymphatic system, and affect a systemic immune response against tumor cells far removed from the prostate. Subtotal cryoablation (rather than total cryoablation) of the prostate is performed in order to allow for the creation of early necrotic prostatic tissue while minimizing the likelihood of freezing other, non-prostatic structures such as the neuro-vascular bundles, the anterior rectal wall, and other uninvolved bowel.

Immediately prior to the cryoablation procedure, four cryopreserved vials containing the patient's cultured dendritic cells are thawed to room temperature. The cell preparation should be thawed for a total of less than about 60 minutes prior to injection. The cryopreserved cell preparation typically requires about 15 to 30 minutes to thaw at ambient temperature. While the cryoablation procedure proceeds, a laboratory technician injects 0.5 ml sterile saline into each thawed vial, using a 1.0 cc syringe equipped with a 10 to 15 cm 18 gauge hypodermic needle. The contents of each of the four vials are then gently drawn into each of four syringes and stored at room temperature until the completion of the cryoablation procedure.

For the cryoablation procedure, the latest generation Endocare Cryocare CS® system is employed. The CS system uses liquid argon gas as a cryogen, and liquid helium as a warming agent. Through thermocouple feedback, this system allows for controlled freezing to the volume of prostate tissue targeted, and “active” thawing of the same volume, either at the discretion of the physician or automatically via use of a computer-mediated system. In addition, the Cryocare CS system employs integrated ultrasound, which allows the operator to monitor all aspects of planning, probe placement and the progress of the freezing event via ultrasound on one unit.

The patient is placed in the dorsal lithotomy position, and the perineum washed in Betadine® solution and draped with an adhesive drape. Prophylactic ciprofloxacin is administered intravenously. A transperineal brachytherapy-style grid is placed against the perineum over the prostate and the transrectal ultrasound probe is inserted into the rectum. Following induction of spinal anesthesia, the operator establishes the position of the prostate superiorly (base) and inferiorly (apex) using the sagittal mode of the ultrasound transducer and then orients the probe mid-gland in transverse view.

Following successful placement of the grid and ultrasound probe, the placement of 3 mm cryoprobes and thermocouples commences. In order to produce the sub-total cryoablation, four cryoprobes from the CS system are introduced into the prostate under ultrasound guidance, one in each quadrant of the prostate as considered transversely. Transverse ultrasound mode will be used to establish placement of the cryoprobes in each transverse quadrant; sagittal mode will be used to establish the placement of the tip of the cryoprobes at the prostate-vesical interface.

Once the cryoprobes have been placed, the operator places five thermocouples under ultrasound guidance. Three thermocouples are placed posteriorly: two postero-laterally in the gland (one each on the right and left) near the putative location of the neuro-vascular bundles, and one in the prostate parenchyma immediately anterior to the rectal wall in the midline. The remaining two thermocouples are placed in the antero-lateral aspect of the gland, one left and one right. Following the placement of cryoprobes and thermocouples, proper placement will once again be verified by the operator. Upon verification, the freezing process can proceed.

Using the control panel mounted on the Cryocare CS system, the operator initiates tissue freezing. The attached system video monitor displays the temperatures at each probe and thermocouple on a schematic transverse prostate section. On the attached ultrasound monitor, evidence of the emerging “iceball” will be apparent as a hyperechoic edge leading outward from an echoless (black) circle.

Care must be taken to keep the temperature at all thermocouples greater than −10° C. Once the volume of frozen prostate as judged by hyperechoic iceball ridge is sufficient and all thermocouple temperatures are greater than −10° C., the thaw function of the Cryocare CS system in invoked, and, as a result, helium flows through the probes, warming the tissue as can be verified and monitored by the thermocouple and cryoprobe temperature outputs on the system video monitor. On ultrasound, the non-echoic ice is replaced by ultrasound signal through the four thawing zones.

Once all system components register body temperature (37° C.), the freezing process and thawing process is repeated, thus achieving a double freeze with double active thaw. Once body temperature is again established in the previously frozen regions, the cryoprobes and thermocouples are removed through the perineal template and discarded. The ultrasound probe and template are kept in place.

For each of the four syringes prepared prior to the cryoablation procedure, the syringe is held by the operator and the needle introduced through the perineal template in a coordinate that correlates with each of the four previously frozen zones. Care must be taken to not use the puncture wound created by any of the cryoprobes or thermocouples, as these wounds may allow for loss of dendritic cell injection product.

The thawed tissue should still be visible on the ultrasound monitor as the ultrasound waves will reflect differently on these areas than on the surrounding, unfrozen tissue. Using sagittal mode, the operator places the needle through the prostate almost—but not to—the prostate-vesical junction. By depressing the plunger and withdrawing the syringe, the operator deposits the syringe contents along previously frozen zones created by the cryoablation procedure.

Once all four dendritic cell preparations have been introduced into all four frozen tissue zones, the perineal template is removed, the ultrasound probe removed, and the perineum bandaged. The patient is then transferred to a post-anesthesia unit for recovery.

### Example 3

### Treatment of Human Malignant Melanoma by Radiotherapy and Intratumoral Injection of DCs

Human malignant melanoma is often highly metastatic and radioresistant (Weichselbaum, et al., Proc. Natl. Acad. Sci. USA 82:4732-4735 (1985); Rubin, P. (1993) Clinical Oncology: A Multidisciplinary Approach for Physicians and Students 7th Ed, Vol. 306, 72 W. B. Saunders Philadelphia), however, ionizing radiation has shown therapeutic benefits. Ionizing radiation is a portion of the high energy electromagnetic radiation spectrum which can penetrate and be transmitted through tissues. A melanoma patient is subjected to ionizing radiation treatment, following standard protocol. The level of melanoma antigens, including Melan-A/MART-1, MAGE, NY-ESO-1, is monitored following irradiation. For a more detailed list of tumor antigens which may be additionally or alternatively monitored, see, e.g. Urban and Schreiber, Annu Rev. Immunol. 10:617-44 (1992), and Renkvist, et al., Cancer Immunol. Immunother. 50(1):3-15 (2001). A dendritic cell preparation, prepared as described above, is then introduced intratumorally, at a time when the level of melanoma-specific antigens is at or near the maximum value, and the efficacy of treatment is monitored.

### Example 4

### Treatment of Breast Cancer by Chemotherapy and Intratumoral Injection of DCs

A patient with hormone-sensitive, node-positive early breast cancer is treated with standard chemotherapy (cyclophosphamide, methotrexate and 5-fluorouracil (CMF)). During and following chemotherapy, the level of tumor-specific antigens, including one or more of carcinoembryonic antigen, NY-BR-1, NY-ESO-1, MAGE-1, MAGE-3, BAGE, GAGE, SCP-1, SSX-1, SSX-2, SSX-4, CT-7, Her2/neu, NY-Br-62, NY-Br-85, and tumor protein D52, is monitored. A dendritic cell preparation, prepared as described above, is then introduced intratumorally or proximate to the cancer, at a time when the level of tumor-specific antigen(s) is at or near the maximum value, and the efficacy of treatment is monitored.

The patent and scientific publications cited herein reflect the general level of skill in the field and are hereby incorporated by reference herein in their entireties for all purposes and to the same extent as if each was specifically and individually indicated to be incorporated by reference. In the case of any conflict between a cited reference and this specification, this specification shall control.

While the present invention has been described in the context of the embodiments illustrated and described herein, the invention may be embodied in other specific ways or in other specific forms without departing from its spirit or essential characteristics. Therefore, the described embodiments are to be considered in all respects as illustrative and not restrictive. The scope of the invention is, therefore, indicated by the appended claims rather than by the foregoing description, and all changes that come within the meaning and range of equivalency of the claims are to be embraced within their scope.

What is claimed is:

1. A method of treating a human subject diagnosed with prostate tumor wherein said treatment consists essentially of the consecutive steps of:
(a) isolating dendritic cell precursors from said human subject;
(b) culturing and differentiating said dendritic cell precursors ex vivo, without a maturation step;
(c) subjecting said prostate tumor or cancerous prostate tissue in said human subject to cellular distress resulting in necrosis, apoptosis, or osmotic cellular injury, and the liberation of tumor specific antigen PSA;
(d) subjecting said human subject to dendritic cell therapy consisting of delivering at least about 7-10×10⁶ non-loaded differentiated autologous dendritic cells not subjected to ex vivo maturation from step (b), in the absence of ex vivo matured dendritic cells, into or proximate to said prostate tumor or cancerous prostate tissue subjected to cellular distress, whereby the dendritic cells uptake said PSA antigen; and
(e) allowing the systemic immune response to occur against the prostate tumor or cancerous prostate tissue, thereby treating said human subject.

2. The method of claim 1, wherein inflicting said cellular distress results in the release of one or more inflammatory factors.

3. The method of claim 2, wherein the inflammatory factors comprise at least one of TNF-α and IL-1β.

4. The method of claim 1, wherein the cellular distress results from one or more of cryotherapy, heat ablation, chemotherapy, radiation therapy, and ultrasound therapy applied against at least a portion of the tumor or cancerous tissue.

5. The method of claim 1 wherein intratumoral delivery is performed by intratumoral injection of said dendritic cells.

6. The method of claim 1, wherein intratumoral delivery is performed through the vasculature of said tumor.

7. The method of claim 1, wherein said tumor is part of an organ.

8. The method of claim 7, wherein intratumoral delivery is performed through direct perfusion of said organ.
